(19) United States
(12) Patent Application Publication
(10) Pub. No.: US 2025/0002441 A1
(43) Pub. Date: Jan. 2, 2025

(54) METHOD FOR PRODUCING ETHYL ACETATE

(71) Applicant: Resonac Corporation, Tokyo (JP)

(72) Inventors: Shintaro ITAGAKI, Oita-shi (JP); Takuro SASAKI, Oita-shi (JP); Yasuhiro HOSOGI, Oita-shi (JP); Yasuhiro IWAMA, Oita-shi (JP)

(73) Assignee: Resonac Corporation, Tokyo (JP)

(21) Appl. No.: 18/694,549

(22) PCT Filed: Oct. 24, 2022
(86) PCT No.: PCT/JP2022/039570

§ 371 Date: Mar. 22, 2024

(30) Foreign Application Priority Data
Dec. 15, 2021 (JP) 2021-203661

(51) Int. Cl. C07C 67/04; B01J 21/08; B01J 23/652; B01J 37/02

(52) U.S. Cl. CPC C07C 67/04; B01J 21/08; B01J 23/6527; B01J 37/0201; B01J 37/0236

(57) ABSTRACT

A method for producing ethyl acetate by reacting ethylene with acetic acid is provided in which side reactions are inhibited from proceeding and a continuous stable operation is possible over a long period. The method for producing ethyl acetate comprises reacting ethylene with acetic acid in the presence of a catalyst comprising a support and, fixed thereto, a heteropolyacid or a salt thereof, the catalyst having a palladium concentration in the range of 0.1-14 mass ppb.

### TECHNICAL FIELD

The present invention relates to a method for producing ethyl acetate using a catalyst in which a heteropolyacid or a salt thereof is supported on a carrier.

### BACKGROUND

It is well known that a corresponding ester can be prepared from a lower aliphatic carboxylic acid and a lower olefin by a gas phase catalytic reaction. In this reaction, it is also well known that a supported catalyst in which a heteropolyacid or a salt thereof is supported on a carrier is useful (Patent Literature 1).

In an industrial production process using a catalyst, there is a problem that an impurity derived from a raw material, by-products produced by a reaction, etc., may cause deterioration of the catalyst, which in turn may lead to deterioration of the reaction results. In particular, when the operation is continuously carried out in a process having a circulation system, there is a problem, such as falling into a vicious cycle in which various impurities and by-products accumulate in the system, the deterioration of a catalyst progresses due to the effect of these impurities and by-products, and further side reactions are promoted.

As a solution for suppressing catalyst deterioration in a synthesis reaction of esters using a supported heteropolyacid catalyst, it is known that acetylenes, halogens, aldehydes, basic nitrogen compounds, and metals or metal compounds contained in a feedstock are substantially free (Patent Literatures 2 to 6).

In a catalytic reaction, the reaction may proceed with low levels of noble metals, on the order of ppm by mass or ppb by mass, contained in a raw material, laboratory equipment, or industrial-scale manufacturing equipment. For example, Non-Patent Literature 1 describes that the Suzuki-Miyaura coupling reaction proceeds with palladium on the order of ppb by mass contained in sodium carbonate. Non-Patent Literature 2 describes that the Suzuki-Miyaura coupling reaction proceeds with an extremely small amount of metal contained in a used PTFE stirrer.

### CITATION LIST

### Patent Literature

[PTL 1] JP H09-118647 A
[PTL 2] JP 2004-18404 A
[PTL 3] JP 2004-83473 A
[PTL 4] JP H11-269126 A
[PTL 5] JP 2002-520380 A
[PTL 6] JP 2002-520381 A

### Non-Patent Literature

[NPL 1] The Journal of Organic Chemistry, 70 volumes, p.161-168 (2005).
[NPL 2] ACS Catalysis, 9 volumes, p. 3070-3081 (2019).

### SUMMARY OF INVENTION

### Technical Problem

In order to suppress an unexpected reaction caused by an extremely small amount of noble metal contamination, it is necessary to control a noble metal component contained in a catalyst, etc., in addition to the components in a feedstock described in the prior art. However, the effect of a small amount of a noble metal component as a catalyst poison in a heteropolyacid catalyst system used for the production of ethyl acetate in which ethylene and acetic acid are reacted has hitherto been unknown.

The present invention provides a method for producing ethyl acetate in which ethylene and acetic acid are reacted, which is capable of suppressing the progress of side reactions and operating continuously and stably for a long period of time.

### Solution to Problem

As a result of intensive studies, the present inventors have found that, in a method for producing ethyl acetate in which ethylene and acetic acid are reacted in the presence of a catalyst in which a heteropolyacid or a salt thereof is supported on a carrier, by controlling the palladium content in the catalyst to be in the range of 0.1 to 14 ppb by mass, the progress of side reactions is suppressed, and thus a stable operation can be carried out continuously for a long period of time, thereby completing the present invention.

That is, the present invention relates to the following matters [1] to [4].

[1]

A method for producing ethyl acetate by reacting ethylene with acetic acid in the presence of a catalyst in which a heteropolyacid or a salt thereof is supported on a carrier,
wherein the palladium content in the catalyst is in the range of 0.1 to 14 ppb by mass.
   [2]

The method for producing ethyl acetate according to [1], wherein the heteropolyacid is silicotungstic acid or phosphotungstic acid.

[3]

The method for producing ethyl acetate according to [1] or [2], wherein the carrier is silica.

[4]

The method for producing ethyl acetate according to any one of [1] to [3], comprising a step of measuring the palladium content in the catalyst before the reaction.

### Advantageous Effects of Invention

According to the present invention, it is possible to provide a method for producing ethyl acetate in which ethylene and acetic acid are reacted, which is capable of suppressing the progress of side reactions and continuously and stably operating for a long period of time.

### DESCRIPTION OF EMBODIMENTS

Hereinafter, preferred embodiments of the present invention will be described. However, it should be noted that the present invention is not limited to these embodiments only, and therefore various applications can be made within the spirit and practice of the present invention.

A method for producing ethyl acetate according to one embodiment is a method for producing ethyl acetate by reacting ethylene with acetic acid in the presence of a catalyst in which a heteropolyacid or a salt thereof is supported on a carrier, wherein the catalyst having a specific palladium content is used.

### <Catalyst for Producing Ethyl Acetate>

### [Preparation of Catalyst for Producing Ethyl Acetate]

In one embodiment, ethyl acetate is prepared by reacting ethylene with acetic acid in the gas phase using a solid acid catalyst. As the solid acid catalyst for producing ethyl acetate, one comprising a heteropolyacid or a salt thereof (also referred to as a “salt of a heteropolyacid” in the present disclosure) as a major active component of the catalyst is used, wherein the heteropolyacid or the salt thereof is supported on a carrier. In the present disclosure, one in which a heteropolyacid or a salt thereof is supported on a carrier is simply referred to as a “catalyst”.

### [Heteropolyacid and Salt Thereof]

A heteropolyacid is an acid composed of a central element and a peripheral element to which oxygen is bonded. The central element is usually silicon or phosphorus, but can be selected from any one selected from a wide variety of elements of Groups 1 to 17 of the Periodic Table of the Elements.

Examples of the central element constituting the heteropolyacid include a cupric ion; divalent ions of beryllium, zinc, cobalt and nickel; trivalent ions of boron, aluminum, gallium, iron, cerium, arsenic, antimony, phosphorus, bismuth, chromium and rhodium; tetravalent ions of silicon, germanium, tin, titanium, zirconium, vanadium, sulfur, tellurium, manganese, nickel, platinum, thorium, hafnium and cerium, and other tetravalent rare earth ions; pentavalent ions of phosphorus, arsenic, vanadium and antimony; a hexavalent ion of tellurium; and a heptavalent ion of iodine, but are not limited thereto.

Specific examples of the peripheral element include tungsten, molybdenum, vanadium, niobium, and tantalum, but are not limited thereto.

Such heteropolyacids are known as “polyoxoanions”, “polyoxometalates” or “metal oxide clusters”. The structures of some of the well-known anions are named after the researchers in this field, and for example, the Keggin structure, the Wells-Dawson structure and the Anderson-Evans-Perloff structure are known. For details, the description in “Chemistry of Polyacids” (edited by the Chemical Society of Japan, Quarterly Chemical Review No. 20, 1993) can be referred to. A heteropolyacid usually has a high molecular weight, e.g., a molecular weight in the range of 700 to 8,500, and includes not only a monomer thereof but also a dimeric complex thereof.

The salt of the heteropolyacid is not particularly limited as long as it is a metal salt or an onium salt in which some or all of the hydrogen atoms of the aforementioned heteropolyacid are substituted. Examples of the salt include metal salts of lithium, sodium, potassium, cesium, magnesium, barium, copper, silver and gallium, and onium salts, such as ammonium salts, but are not limited thereto.

Examples of the heteropolyacid that can be used in the catalyst include:
silicotungstic acid: H₄[SiW₁₂O₄₀]·xH₂O
phosphotungstic acid: H₃[PW₁₂O₄₀]·xH₂O
phosphomolybdic acid: H₃[PMo₁₂O₄₀]·xH₂O
silicomolybdic acid: H₄[SiMo₁₂O₄₀]·xH₂O
silicovanadotungstic acid: H₄+n[SiVₙW₁₂₋ₙO₄₀]·xH₂O
phosphovanadotungstic acid: H₃+n[PVₙW₁₂₋ₙO₄₀]·xH₂O
phosphovanadomolybdic acid: H₃+n[PVₙMo₁₂₋ₙO₄₀]·xH₂O
silicovanadomolybdic acid: H₄+n[SiVₙMo₁₂₋ₙO₄₀]·xH₂O
silicomolybdotungstic acid: H₄[SiMoₙW₁₂₋ₙO₄₀]·xH₂O
phosphomolybdotungstic acid: H₃[PMoₙW₁₂₋ₙO₄₀]·xH₂O
   wherein n is an integer of 1 to 11 and x is an integer greater than or equal to 1, but are not limited thereto.

The heteropolyacid is preferably silicotungstic acid, phosphotungstic acid, phosphomolybdic acid, silicomolybdic acid, silicovanadotungstic acid or phosphovandotungstic acid, and more preferably silicotungstic acid or phosphotungstic acid.

There is no particular limitation on the method for synthesizing such a heteropolyacid, and any methods may be used. For example, a heteropolyacid can be obtained by heating an acidic aqueous solution (approximately pH1 to pH2) containing a salt of molybdic acid or tungstic acid and a simple oxoacid of a heteroatom or a salt thereof. A heteropolyacid compound can be isolated, for example, by crystallization separation as a metal salt from the produced aqueous heteropolyacid solution.

Specific examples of the manufacture of the heteropolyacid are described on page 1413 of “New Experimental Chemistry 8, Synthesis of Inorganic Compound (III)” (edited by the Chemical Society of Japan, published by Maruzen Co., Ltd., Aug. 20, 1984, third edition), but are not limited thereto. The structural confirmation of the synthesized heteropolyacid can be carried out by chemical analysis as well as X-ray diffraction, UV, or IR spectra measurements.

Preferred examples of the salt of the heteropolyacid include lithium salts, sodium salts, potassium salts, cesium salts, magnesium salts, barium salts, copper salts, silver salts, gallium salts, and ammonium salts of the aforementioned preferred heteropolyacids.

Specific examples of the salt of the heteropolyacid include a lithium salt of silicotungstic acid, a sodium salt of silicotungstic acid, a cesium salt of silicotungstic acid, a copper salt of silicotungstic acid, a silver salt of silicotungstic acid, a gallium salt of silicotungstic acid; a lithium salt of phosphotungstic acid, a sodium salt of phosphotungstic acid, a cesium salt of phosphotungstic acid, a copper salt of phosphotungstic acid, a silver salt of phosphotungstic acid, a gallium salt of phosphotungstic acid; a lithium salt of phosphomolybdic acid, a sodium salt of phosphomolybdic acid, a cesium salt of phosphomolybdic acid, a copper salt of phosphomolybdic acid, a silver salt of phosphomolybdic acid, a gallium salt of phosphomolybdic acid; a lithium salt of silicomolybdic acid, a sodium salt of silicomolybdic acid, a cesium salt of silicomolybdic acid, a copper salt of silicomolybdic acid, a silver salt of silicomolybdic acid, a gallium salt of silicomolybdic acid; a lithium salt of silicovanadotungstic acid, a sodium salt of silicovanadotungstic acid, a cesium salt of silicovanadotungstic acid, a copper salt of silicovanadotungstic acid, a silver salt of silicovanadotungstic acid, a gallium salt of silicovanadotungstic acid; a lithium salt of phosphovanadotungstic acid, a sodium salt of phosphovanadotungstic acid, a cesium salt of phosphovanadotungstic acid, a copper salt of phosphovanadotungstic acid, a silver salt of phosphovanadotungstic acid, a gallium salt of phosphovanadotungstic acid; a lithium salt of phosphovanadomolybdic acid, a sodium salt of phosphovanadomolybdic acid, a cesium salt of phosphovanadomolybdic acid, a copper salt of phosphovanadomolybdic acid, a silver salt of phosphovanadomolybdic acid, a gallium salt of phosphovanadomolybdic acid; a lithium salt of silicovanadomolybdic acid, a sodium salt of silicovanadomolybdic acid, a cesium salt of silicovanadomolybdic acid, a copper salt of silicovanadomolybdic acid, a silver salt of silicovanadomolybdic acid, and a gallium salt of silicovanadomolybdic acid.

The salt of the heteropolyacid is preferably a lithium salt of silicotungstic acid, a sodium salt of silicotungstic acid, a cesium salt of silicotungstic acid, a copper salt of silicotungstic acid, a silver salt of silicotungstic acid, a gallium salt of silicotungstic acid; a lithium salt of phosphotungstic acid, a sodium salt of phosphotungstic acid, a cesium salt of phosphotungstic acid, a copper salt of phosphotungstic acid, a silver salt of phosphotungstic acid, a gallium salt of phosphotungstic acid; a lithium salt of phosphomolybdic acid, a sodium salt of phosphomolybdic acid, a cesium salt of phosphomolybdic acid, a copper salt of phosphomolybdic acid, a silver salt of phosphomolybdic acid, a gallium salt of phosphomolybdic acid; a lithium salt of silicomolybdic acid, a sodium salt of silicomolybdic acid, a cesium salt of silicomolybdic acid, a copper salt of silicomolybdic acid, a silver salt of silicomolybdic acid, a gallium salt of silicomolybdic acid; a lithium salt of silicovanadotungstic acid, a sodium salt of silicovanadotungstic acid, a cesium salt of silicovanadotungstic acid, a copper salt of silicovanadotungstic acid, a silver salt of silicovanadotungstic acid, a gallium salt of silicovanadotungstic acid; a lithium salt of phosphovanadotungstic acid, a sodium salt of phosphovanadotungstic acid, a cesium salt of phosphovanadotungstic acid, a copper salt of phosphovanadotungstic acid, a silver salt of phosphovanadotungstic acid, or a gallium salt of phosphovanadotungstic acid.

As the salt of the heteropolyacid, a lithium salt of silicotungstic acid or a cesium salt of phosphotungstic acid is particularly preferable.

### [Carrier]

The carrier is not particularly limited, and a porous material commonly used as a carrier for a catalyst can be used. Examples of preferred carriers include silica, alumina, silica-alumina, diatomaceous earth, montmorillonite, titania and zirconia, with silica being more preferred.

The carrier preferably has a specific surface area measured by the BET method in the range of 10 to 1000 m2/g, and more preferably in the range of 100 to 500 m2/g.

The bulk density of the carrier is preferably in the range of 50 to 1000 g/L, and more preferably in the range of 300 to 500 g/L. In the present disclosure, the bulk density of the carrier is a value calculated from the mass of the carrier and the volume of a graduated cylinder, in which the carrier is put into the glass graduated cylinder in several parts, and the graduated cylinder containing the carrier is tapped every time the carrier is put into the glass graduated cylinder, so that the carrier is put into the graduated cylinder until it reached a measurement volume of the graduated cylinder.

The water absorption rate of the carrier is preferably 0.05 to 3 g-water/g-carrier, and more preferably 0.1 to 2 g-water/g-carrier.

With respect to the pore structure of the carrier, the average pore diameter thereof is preferably in the range of 1 to 1000 nm, and more preferably in the range of 2 to 800 nm. When the average pore diameter is 1 nm or more, gas diffusion can be facilitated. When the average pore diameter is 1000 nm or less, a specific surface area of the carrier which is necessary for obtaining catalytic activity can be ensured.

There is no particular limitation on the shape of the carrier. Specific examples thereof include a powder, a sphere, and a pellet. The optimum shape can be selected depending on the reaction type and reactor to be used, etc.

There is no particular limitation on the particle size of the carrier. When the carrier is spherical, the particle diameter thereof is preferably in the range of 1 to 10 mm, and more preferably in the range of 2 to 8 mm. When the reaction is carried out by filling the reaction tube with the catalyst, the particle diameter being 1 mm or more can prevent excessive increase in pressure loss when the gas flows, so that effective gas circulation is ensured. The particle diameter being 10 mm or less facilitates diffusion of the raw material gas into the inside of the catalyst, so that the catalytic reaction can effectively proceed.

In one embodiment, a method for supporting a heteropolyacid or a salt thereof on a carrier includes a step of making the carrier absorb (impregnate) an aqueous solution of the heteropolyacid or the salt thereof (an aqueous solution of the heteropolyacid) (an impregnation step), and a step of drying the carrier impregnated with the aqueous solution of the heteropolyacid under specific drying conditions (a drying step) in this order. Although other steps (for example, an air drying step, a transfer step from the impregnation apparatus to the drying apparatus, etc.) may be included between the impregnation step and the drying step, the two steps are preferably carried out continuously.

### [Palladium Contained in Catalyst]

The form of palladium contained in the catalyst is not particularly limited, and examples thereof include metallic palladium, palladium oxide, an inorganic salt of palladium, and a palladium complex. Palladium may be unintentionally incorporated during catalyst preparation. For example, this is the case when a catalyst using palladium is prepared, and then a catalyst of one embodiment is prepared using the same apparatus.

The content of palladium contained in the catalyst (content of a palladium atom) is in the range of 0.1 to 14 ppb by mass, preferably in the range of 0.5 to 12 ppb by mass, and more preferably in the range of 1 to 10 ppb by mass. When the palladium content exceeds 14 ppb by mass, catalytic deterioration may proceed due to side reactions. The content is based on the total mass of the catalyst including the heteropolyacid or the salt thereof and the carrier.

A method for quantitatively analyzing palladium contained in the catalyst at a mass ppb-level includes a method in which the catalyst is calcined in an oxidizing atmosphere, then palladium is extracted from the obtained calcined body into an acidic solution, and the noble metal content in the obtained extract is measured by ICP mass spectrometry. Specifically, the method is carried out by the analytical operation described in the section of Comparative catalyst F used in Comparative Example described later.

### <Preparation of Ethyl Acetate>

In one embodiment, ethyl acetate can be obtained by reacting acetic acid with ethylene in a gas phase using a solid acid catalyst in which a heteropolyacid or a salt thereof is supported on a carrier. In one embodiment, a method for producing ethyl acetate preferably comprises a step of measuring the content of palladium in a catalyst before the reaction of acetic acid and ethylene, and a catalyst having a content of palladium in the range of 0.1 to 14 ppb by mass is used in the reaction.

Acetic acid and ethylene are preferably diluted with an inert gas, such as nitrogen gas in terms of reaction heat removal. Specifically, a gas containing acetic acid and ethylene as a raw material is flown through a vessel filled with a solid acid catalyst, and the gas is brought into contact with the solid acid catalyst, whereby they can be reacted.

It is preferable to add a small amount of water to the gas containing acetic acid and ethylene as a raw material from the viewpoint of maintaining the catalytic activity, and in one embodiment, the reaction is carried out in the presence of water vapor. However, when a too large amount of water is added, the amount of by-products, such as an alcohol, and an ether may increase. The added amount of water is preferably 0.5 to 15 mol %, and more preferably 2 to 8 mol %, in terms of a molar ratio of water to the total of acetic acid, ethylene, and water.

The ratio of ethylene to acetic acid as a raw material is not particularly limited, but the molar ratio of ethylene to acetic acid is preferably in the range of ethylene:acetic acid=1:1 to 40:1, more preferably in the range of 3:1 to 20:1, and still more preferably in the range of 5:1 to 15:1.

The reaction temperature is preferably in the range of 50° C. to 300° C., and more preferably in the range of 140° C. to 250° C. The reaction pressure is preferably in the range of 0 PaG to 3 MPaG (gauge pressure), and more preferably in the range of 0.1 MPaG to 2 MPaG (gauge pressure). In one embodiment, the reaction temperature is 150 to 170° C., and the reaction pressure is 0.1 to 2.0 MPaG (gauge pressure).

SV (gas space velocity) of the gas containing the raw material is not particularly limited, but when it is too large, the raw material may pass through without reaction progressing sufficiently, while when it is too small, productivity may be lowered. SV (the volume of a raw material passing through 1 L of a catalyst for one hour (L/L·h=h⁻¹)) is preferably 500 to 20000 h⁻¹, and more preferably 1000 to 10000 h⁻¹.

### EXAMPLES

Although the present invention will be further described with reference to the following Examples and Comparative Examples, the present invention is not limited to these examples.

### [Bulk Density Measurement of Silica Carrier]

A silica carrier was put into a tared glass graduated cylinder in several parts, and the graduated cylinder containing the carrier was tapped every time the silica carrier was put into the graduated cylinder, so that the carrier was put into the graduated cylinder until it reached a measurement volume of the graduated cylinder. Then, the mass of the graduated cylinder containing the carrier was measured, and the bulk density of the carrier was determined based on the tare and volume of the graduated cylinder.

### [Preparation of Catalyst A]

In 75.8 g (75.8 mL) of pure water, 120 g of a commercially available Keggin silicotungstic acid 24 hydrate (H₄SiW₁₂O₄₀·24H₂O, Nippon Inorganic Chemical Industry Co., Ltd.), and 0.003 mg of palladium nitrate (Pd (NO₃)₂, FUJIFILM Wako Pure Chemical Corporation) were dissolved to prepare an aqueous solution of 108 mL. The resulting aqueous solution was then added to 0.3 L (134 g) of a commercially available silica carrier (spherical, diameter: about 5 mm, bulk density: 451 g/L), and stirred well to be impregnated in the carrier, such that silicotungstic acid was supported on the silica carrier. The silica carrier on which silicotungstic acid was supported was transferred to a porcelain dish, air-dried for one hour, and then dried for 1 hour in a ventilated box type hot air dryer (experimental ventilated shelf type dryer, model name: LABO-4CS, Nagato Denki MFG. Co., Ltd.) in which the temperature of hot air was set to 120° C. and the wind speed was set to 50 m/min, thereby obtaining catalyst A (Pd content calculated from the charge: 5 ppb by mass).

### [Preparation of Catalyst B]

Catalyst B (Pd content calculated from the charge: 7 ppb by mass) was obtained in the same manner as catalyst A, except that the amount of palladium nitrate used was changed to 0.004 mg.

### [Preparation of Comparative Catalyst C]

Comparative catalyst C (Pd content calculated from the charge: 15 ppb by mass) was obtained in the same manner as catalyst A, except that the amount of palladium nitrate used was changed to 0.008 mg.

### [Preparation of Comparative Catalyst D]

Comparative catalyst D (Pd content calculated from the charge: 25 ppb by mass) was obtained in the same manner as catalyst A, except that the amount of palladium nitrate used was changed to 0.014 mg.

### [Preparation of Reference Catalyst E]

Reference catalyst E (Pd content calculated from the charge: 0 ppb by mass) was obtained in the same manner as catalyst A, except that palladium nitrate was not used.

### [Comparative Catalyst F]

About 300 kg of catalyst F was produced in an actual plant in such a manner that the amount ratio of the commercially available Keggin silicotungstic acid 24 hydrate (H₄SiW₁₂O₄₀·24H₂O, Nippon Inorganic Chemical Industry Co., Ltd.) to the commercially available silica carrier (spherical, diameter: about 5 mm, bulk density: 451 g/L) was the same as that of catalyst E without palladium nitrate. The palladium content in catalyst F was measured to be 23 ppb by mass. In the producing process of catalyst F, it is presumed that a very small amount of palladium component was mixed in for some reason. The palladium content was measured by the following method.

### (Measurement of Palladium Content)

Catalyst F was pulverized in an agate mortar, and 2 g of the powder was filled in a capped alumina crucible and calcined in a muffle furnace at 900° C. for 3 hours under air circulation.

0.1 g of the calcined sample was placed in a quartz beaker, and then 3 mL of an ultrapure water, 1 mL of 68% by mass aqueous nitric acid solution (HNO₃; Tama Chemicals Co., Ltd., TAMAPURE-AA-100), and 1 mL of 30% by mass hydrochloric acid (HCl; Tama Chemicals Co., Ltd., TAMAPURE-AA-100) were added. The content was heated on a hot plate set at 100° C. for 2 hours with each shaking. After the content was cooled, 5 mL of an ultrapure water was added. The solution was then filtered through a 0.45 μm disposable filter and collected in a polypropylene vessel. The quartz beaker was washed with 10 mL of an ultrapure water, the washing liquid was filtered through a 0.45 μm disposable filter, and the filtrate was collected in the polypropylene vessel. The washing operation was carried out three times in total.

The filtrate collected in the polypropylene vessel was adjusted to 50 mL, and the palladium content in the solution was determined by ICP mass spectrometry. The palladium content (ppb by mass) in Comparative catalyst F was calculated from the palladium content analysis value and the mass of Comparative catalyst F charged. In ICP mass spectrometry, 7700 manufactured by Agilent Technologies was used to quantify the palladium content by a calibration curve method. Palladium was measured in helium mode and quantified using m/z=105.

### [Preparation of Ethyl Acetate]

40 mL of the catalyst was charged into a stainless-steel reaction tube having an inner diameter of 25 mm, which was then pressurized to 0.75 MPaG (gauge pressure), and then heated to 155° C. A mixed gas of nitrogen gas 85.5 mol %, acetic acid 10.0 mol %, and water 4.5 mol % was processed under the condition of SV (the volume of a raw material passing through 1 L of a catalyst for one hour (L/L·h=h⁻¹))=1500 h⁻¹ for 30 minutes, and then a mixed gas of ethylene 78.5 mol %, acetic acid 10 mol %, water 4.5 mol %, and nitrogen gas 7.0 mol % was introduced and reacted under the condition of SV=1500 h⁻¹.

The reaction was carried out with adjusting the reaction temperature so that the portion having the highest temperature among the portions obtained by dividing the catalyst layer into 10 portions was 165.0° C. The gas passed through during a predetermined period of time from the start of the reaction was condensed with cooling water and collected (hereinafter, this is referred to as “condensate”), and the obtained condensate was analyzed. Further, for the uncondensed gas remaining without condensation (hereinafter, this is referred to as “uncondensed gas”), the gas flow rate was measured for the same time as the condensate, and 100 mL thereof was taken out and analyzed.

### [Analysis Method of Condensate]

The condensate was analyzed by a gas chromatography apparatus. Using the internal standard method, an analysis solution is prepared by adding 1 mL of 1,4-dioxane as an internal standard to 10 mL of the reaction liquid, and 0.2 μL of the analysis solution was injected to carry out the analysis under the following conditions.
Gas chromatography apparatus: Agilent Technologies 7890B
Column: capillary column DB-WAX (length: 30 m, inner diameter: 0.32 mm, film thickness: 0.5 μm)
Carrier gas: nitrogen gas (split ratio: 200:1, column flow rate: 0.8 mL/min)
Temperature conditions: The detector temperature was set to 250° C., the vaporizing chamber temperature was set to 200° C., and the column temperature was held at 60° C. for 5 minutes from the start of the analysis, thereafter, the temperature was raised to 80° C. at a rate of temperature rise of 10° C./min, after reaching 80° C., the temperature was raised to 200° C. at a rate of temperature rise of 30° C./min, and held at 200° C. for 20 minutes.
Detector: FID (H₂ flow rate: 40 mL/min, air flow rate: 450 mL/min)

### [Analysis Method of Uncondensed Gas]

The uncondensed gas was analyzed by a gas chromatography apparatus. 100 mL of the uncondensed gas was collected, and the entire amount was poured into a 500 μL gas sampler attached to the gas chromatography apparatus, and analyzed using an absolute calibration curve method under the following conditions.

### 1. Analysis of Ethyl Acetate

Gas chromatography apparatus: Agilent Technologies 7890A
Column: Agilent J&W GC column DB-624
Carrier gas: He (flow rate: 1.7 mL/min)
Temperature conditions: The detector temperature was set to 230° C., the vaporizing chamber temperature was set to 200° C., and the column temperature was held at 40° C. for 3 minutes from the start of the analysis, and thereafter, the temperature was raised to 200° C. at a rate of temperature rise of 20° C./min.
Detector: FID (H₂ flow rate: 40 m/min, air flow rate: 400 mL/min)

### 2. Analysis of Butene

Gas chromatography apparatus: Agilent Technologies 7890A
Column: SHIMADZU GC GasPro (30 m), Agilent J&W GC column HP-1
Carrier gas: He (flow rate: 2.7 mL/min)
Temperature Conditions: The detector temperature was set to 230° C., the vaporizing chamber temperature was set to 200° C., and the column temperature was held at 40° C. for 3 minutes from the start of the analysis, and thereafter, the temperature was raised to 200° C. at a rate of temperature rise of 20° C./min.
Detector: FID (H₂ flow rate: 40 m/min, air flow rate: 400 mL/min)

### 3. Calculation of Reaction Results

The space time yield of ethyl acetate and the butene selectivity were calculated by the following formulae.

$$Space time yield of ethyl acetate ( g / L \cdot h ) = ( Mass of ethyl acetate produced per hour ) / ( Volume of catalyst used )$$

$$Butene selectivity ( \% ) = ( Number of moles of produced butene / Number of moles of fed ethylene ) \times 100$$

### Example 1

The stainless steel reaction tube (gas phase flow reactor) was charged with 40 mL of catalyst A, and a synthetic reaction of ethyl acetate was carried out. After 5 hours and 200 hours of the reaction, the condensate and the uncondensed gas were analyzed, and the space time yield of ethyl acetate and the butene selectivity were calculated. The results are shown in Table 1.

### Example 2

The synthesis reaction of ethyl acetate was carried out in the same manner as in Example 1, except that catalyst B was used instead of catalyst A, and the analysis was carried out in the same manner as in Example 1. The results are shown in Table 1.

### Comparative Examples 1 to 3

The synthesis reaction of ethyl acetate was carried out in the same manner as in Example 1, except that Comparative catalyst C, Comparative catalyst D, and Comparative catalyst F was used respectively instead of catalyst A, and the analysis was carried out in the same manner as in Example 1. The results are shown in Table 1.

### Reference Example 1

The synthesis reaction of ethyl acetate was carried out in the same manner as in Example 1, except that reference catalyst E was used instead of catalyst A, and the analysis was carried out in the same manner as in Example 1. The results are shown in Table 1.

TABLE 1

| | | | After 5 hours from the | | After 200 hours from the | |
|---|---|---|---|---|---|---|
| | | | start of the reaction | | start of the reaction | |
| | | | Space time | | Space time | |
| | | Palladium | yield of | | yield of | |
| | | content | ethyl | Butene | ethyl | Butene |
| | | (ppb by | acetate | selectivity | acetate | selectivity |
| | Catalyst | mass) | (g/L · h) | (%) | (g/L · h) | (%) |
| Ex. 1 | catalyst A | 5 | 293 | 0.30 | 255 | 0.17 |
| Ex. 2 | catalyst B | 7 | 295 | 0.43 | 251 | 0.15 |
| Comp. | Comparative | 15 | 297 | 0.82 | 236 | 0.17 |
| Ex. 1 | catalyst C | | | | | |
| Comp. | Comparative | 25 | 301 | 0.80 | 235 | 0.16 |
| Ex. 2 | catalyst D | | | | | |
| Comp. | Comparative | 23 | 305 | 0.79 | 239 | 0.18 |
| Ex. 3 | catalyst F | | | | | |
| Ref. | reference | 0 | 295 | 0.14 | 255 | 0.15 |
| Ex. 1 | catalystE | | | | | |

As shown in Table 1, when Examples 1 to 2 are compared with Comparative Examples 1 to 2, it can be seen that the higher the palladium (Pd) content, the higher the butene selectivity after 5 hours from the start of the reaction. Butene, which is one of the main by-products in the present reaction, is a cause of catalyst coking, and therefore, the lower the butene selectivity, the more desirable from the viewpoint of catalyst life.

In Comparative Examples 1 to 2 in which the palladium (Pd) content was high, the reduction rate of the space time yield of ethyl acetate after 200 hours from the start of the reaction was large, and it can be seen that the reduction of the palladium (Pd) content in the catalyst is also superior from the viewpoint of the catalyst life.

Comparing Examples 1 to 2 with Reference Example 1, although the butene selectivity after 5 hours from the start of the reaction is high, the space time yield of ethyl acetate after 200 hours is comparable, it can be seen that the performance is comparable with respect to the lifetime.

Catalyst F of Comparative Example 3 had a palladium content of 23 ppb by mass. Comparative catalyst F has a higher selectivity of butene, which is a by-product, as compared with reference catalyst E.

When a catalyst in which a heteropolyacid or a salt thereof is supported on a carrier is produced using a production apparatus in which palladium may be mixed into the catalyst, it is possible to determine whether the catalyst is suitable for producing ethyl acetate by checking the palladium content of the produced catalyst before the catalyst is used in an actual plant. As a result, it is possible to increase the production efficiency of ethyl acetate in a real plant.

1. A method for producing ethyl acetate by reacting ethylene with acetic acid in the presence of a catalyst in which a heteropolyacid or a salt thereof is supported on a carrier,
wherein the palladium content in the catalyst is in the range of 0.1 to 14 ppb by mass.

2. The method for producing ethyl acetate according to claim 1, wherein the heteropolyacid is silicotungstic acid or phosphotungstic acid.

3. The method for producing ethyl acetate according to claim 1, wherein the carrier is silica.

4. The method for producing ethyl acetate according to claim 1, comprising a step of measuring the palladium content in the catalyst before the reaction.

5. The method for producing ethyl acetate according to claim 2, wherein the carrier is silica.

6. The method for producing ethyl acetate according to claim 2, comprising a step of measuring the palladium content in the catalyst before the reaction.

7. The method for producing ethyl acetate according to claim 3, comprising a step of measuring the palladium content in the catalyst before the reaction.

8. The method for producing ethyl acetate according to claim 5, comprising a step of measuring the palladium content in the catalyst before the reaction.
